(19) United States
(12) Patent Application Publication
(10) Pub. No.: US 2004/0229950 A1
(43) Pub. Date: Nov. 18, 2004

(54) Method and composition with conjugated linoleic acid esters

(76) Inventor: Jack Y. Vanderhoek, Bethesda, MD (US)

Correspondence Address: ANTONELLI, TERRY, STOUT & KRAUS, LLP, 1300 NORTH SEVENTEENTH STREET, SUITE 1800, ARLINGTON, VA, 22209-9889, US

(21) Appl. No.: 10/721,231

(22) Filed: Nov. 26, 2003

Related U.S. Application Data
(60) Provisional application No. 60/428,899, filed on Nov. 26, 2002.

(51) Int. Cl. A61K 31/202

(52) U.S. Cl. 514/560

(57) ABSTRACT

Esters containing conjugated linoleic acids are used to stimulate release of cellular AA, cellular production of prostacyclin (in cells such as endothelial cells) and formation of thromboxane A₂ (in cells such as platelets or endothelial cells or in a subject organism, preferably a mammal).

### BACKGROUND OF THE INVENTION

This application claims the benefit of provisional application U.S. Application Serial No. 60/428,899, filed Nov. 26, 2003, the entire contents of which are incorporated herein by reference.

1. Field of the Invention

The present invention relates to compositions and methods of use relating to esters containing at least one conjugated linoleic acid, particularly, esters containing at least one conjugated linoleic acid selected from the group consisting of 10,12-octadecadienoic acid and 9,11-octadecadienoic acid. More particularly, the present invention relates to compositions and methods of using a lipid containing at least one conjugated linoleic acid, particularly, a lipid containing at least one conjugated linoleic acid selected from the group consisting of 10,12-octadecadienoic acid and 9,11-octadecadienoic acid, to stimulate arachidonic acid release and subsequent enhancement of prostacyclin formation in cells such as endothelial cells and of thromboxane formation in cells such as platelets.

2. Description of the Related Art

Conjugated linoleic acids (CLAs) (notably 9,11- and 10,12-octadecadienoic acids or, more simply, 9,11-18:2 or 10,12-18:2) are isomers of linoleic acid (9,12-linoleic acid). The terms “conjugated linoleic acids” and “CLA” are used interchangeably and in a generalized sense to refer to positional and geometrical isomers of linoleic acid that have a set of conjugated double bonds, rather than non-conjugated double bonds. The conjugated linoleic acids have possible cis (Z) and trans (E) configurations at the double bonds

Conjugated linoleic acids have been found predominantly in meat and dairy products (Chin 1992; Shanta 1992). CLA content is highest in ruminant meats. For example, lamb contains 6 mg of CLAs per gram of fat with smaller amounts being found in poultry and eggs. Dairy products also contain considerable amounts of CLAs. For example, homogenized milk has about 5.5 mg/g of fat.

In the past few years, conjugated linoleic acids have generated considerable interest in cancer and cardiovascular research. A variety of reports have appeared indicating that CLAs may be effective in inhibiting the initiation and/or post-initiation phases of carcinogenesis in several experimental animal models (Ip 1991; Ip 1992; Belury 1995)). CLAs have also been reported as decreasing the incidence of chemically induced skin and forestomach cancers in mice and mammary tumors in rats. Other findings indicate that CLAs have reduced in vitro cell growth when added to malignant melanoma cells, colorectal cancer cells and human breast cancer cells.

As far as the effects of conjugated linoleic acids on cardiovascular disease are concerned, Kritchevsky and co-workers have reported the suppression of atherosclerosis in rabbits (Lee 1994). Thus, when rabbits were fed an atherogenic diet containing CLA, a decrease in total plasma—and LDL cholesterol levels was observed. In another study, Nicolosi found that addition of CLA to the diet of hamsters reduced LDL cholesterol levels and aortic atherosclerosis (Nicolosi 1997).

In U.S. Pat. No. 6,077,525 to Vanderhoek, incorporated herein by reference, it was reported that conjugated linoleic acids inhibit platelet aggregation and formation of thromboxane B₂ (TXB₂), a prostanoid.

Prostanoids are members of the eicosanoid family of metabolites formed from arachidonic acid (AA). Eicosanoids are produced by most mammalian cell types and are potent cellular regulators that function as local mediators since they act at or near the location at which they are synthesized (see, for example, Smith 1996). The two major, hemostatically important, AA metabolites are prostacyclin (PGI₂), produced by large vessel endothelium, and TXA₂, formed by platelets (see for example, Smith 1996). The release of PGI₂ into the bloodstream affects the functions of platelets as well as leukocytes. PGI₂ prevents neutrophil aggegation and chemotaxis and inhibits platelet activation and secretion by raising the platelet cAMP levels (Weksler 1985). In view of its antiaggregatory and vasodilatory activities, PGI₂ is generally considered an antithrombogenic mediator (Weksler 1985) and counteracts the prothrombogenic effects of TXA₂, the major AA metabolite produced by platelets, which is a potent aggregatory and vasoconstrictive agent.

In U.S. Pat. No. 6,077,525, it was reported that conjugated linoleic acids, notably 9,11-18:2 and 10,12-18:2, and hydroxy derivatives thereof, selectively inhibit the conversion of arachidonic acid into thromboxane/prostanoids. As a consequence, the patent disclosed the use of CLAs to inhibit platelet thromboxane formation or platelet aggregation. Further studies have shown that several CLA isomers also inhibited prostacyclin (PGI₂) production in human umbilical vein endothelial cells (HUVECs). All of these studies relate to the use of conjugated linoleic acids in the free fatty acid form.

### SUMMARY OF THE INVENTION

The present invention is based, at least in part, on the finding that prelabeling platelets with 9Z, 11Z-CLA leads to a 2-5-fold enhancement of endogenous platelet release of arachidonic acid (AA) and a 2-4-fold increase in the formation of the platelet eicosanoid thromboxane B₂ (TXB₂). Prelabeling IL-1 βtreated- human umbilical vein endothelial cells (HUVECs) with either 9Z, 11Z-or 10E,12Z-CLA isomers resulted in an 8- and 3-fold (respectively) stimulation of PGI₂ production (as measured by formation of 6-ketoPGF₁ₐ, its inactive, stable metabolite) from endogenous AA. These results are quite intriguing in that the CLA effects on AA metabolism in HUVECs and platelets appear to depend on how CLA is presented to cells, i.e whether as free fatty acid or as an esterified fatty acid. (Prelabeling platelets with CLA results in the incorporation (by esterification) of CLA into the lipid components of these cells, similar to that observed when cells are treated with other fatty acids, see, for example, Spector 1980. It may be expected that the same result may be obtained with prelabeled HUVECs). Furthermore, the ability of such a CLA preparation to stimulate prostacyclin formation would suggest an unexpected antithrombogenic role.

The above-noted selective action of CLAs esterified in lipid form on the release of AA and its conversion to prostanoids acid indicates that the administration of effective amounts of a lipid containing esterified CLA, for example, as an additive to food or in pharmaceutical form, to mammals can provide a useful method for providing antithrombogenic action by stimulating the cyclooxygenase-catalyzed conversion of arachidonic acid to prostacyclins. In a more specific aspect, the invention provides a method for providing antithrombogenic action by administering an effective amount of a CLA ester, particularly a CLA lipid or mixture thereof to a mammal in need of such action. Other aspects of the invention will be evident from the description that follows.

The above and other objects of the invention will become readily apparent to those of skill in the relevant art from the following detailed description and figures, wherein only the preferred embodiments of the invention are shown and described, simply by way of illustration of the best mode of carrying out the invention. As is readily recognized the invention is capable of modifications within the skill of the relevant art without departing from the spirit and scope of the invention.

### BRIEF DESCRIPTION OF THE DRAWINGS

FIG. 1 is a graph showing the comparative effects of 25 μM 9Z, 1 1Z-CLA-, 9E, 11E-CLA-, LA- or SA-treated platelets on platelet TXB₂ production.

FIG. 2 is a graph showing the stimulatory effect of different concentrations of 9Z, 11Z-CLA on HUVEC 6-ketoPGF₁ₐ production.

### DETAILED DESCRIPTION OF THE PREFERRED EMBODIMENTS

The present invention relates to use of esters containing conjugated linoleic acids to stimulate 1) release of cellular AA, 2) cellular production of prostacyclin (in cells such as endothelial cells) and 3) formation of thromboxane A₂ (in cells such as platelets or in a subject organism, preferably a mammal).

The conjugated linoleic acids useful herein may include 7,9-octadecadienoic acid ,11,13-octadecadienoic acid, 9,11-octadecadienoic acid and 10,12-octadecadienpoic acid, as well as other CLA isomers. The above-named CLA isomers may also be called 7,9-18:2,9,11-18:2,10,12-18:2, and 11,13-18:2. The CLAs may be used separately, or in admixture, in either the cis- and/or trans-forms. Most preferred are the 9Z,11Z and the 10E, 12Z isomers.

The compound used in the present invention may be any ester of a conjugated linoleic acid, as described above, and is preferably a lipid. Most preferably, the conjugated linoleic acid ester is a phospholipid, such as, for example, phosphatidylcholine, phosphatidylethanolamine, phosphatidylinositol, phosphatidylserine, cardiolipin and sphingomyelin.

Conjugated linoleic acid esters may be obtained from natural sources or by esterifying free conjugated linoleic acids into lipids presented either in cells or in isolated form. For example, conjugated linoleic acids may be provided to a cell culture or cell-free enzyme system capable of forming lipids using the conjugated linoleic acids.

As indicated, the invention contemplates the addition of the conjugated linoleic acid esters to food or as an active component in a pharmaceutical composition of conventional form, e.g. a tablet, capsule or equivalent. The CLA ester may be added to any type of food, e.g. butter or other spreads, bread or the like. When used in pharmaceutical form, the CLA ester may be the only active component or it may be used in combination with one or more other pharmaceutically effective agents.

The amount of CLA ester used to stimulate can be widely varied depending on other factors, e.g. body weight. However, the amount of CLA ester to be administered can be readily determined for any specific situation. Generally, however, the amount of CLA ester used will be in the range of 0.25 to 0.5 grams as a daily dose per kg of mammal being treated.

The invention is illustrated by the following examples:

### EXAMPLE 1

The following example illustrates the effect of CLA incorporation into platelet lipids on stimulating the release of endogenous AA and increasing the formation of TXB₂. Platelets were prelabeled with either HSA alone or with 25 μM 9Z, 11Z-CLA/HSA for 1 hour at 37° C. After washing, the platelets were incubated for 15 minutes at room temperature, followed by the removal of the supernatants. The supernatants were then assayed for free M content and TXB₂ production. As shown in Table 1, in experiment 1, a 5-fold increase in AA release (27 μg/ml) and a 2-fold increase in TXB₂ formation (0.36 ng/ml) relative to HSA/platelet controls (release of 5.6 μg/ml M, 0.19 ng/ml TXB₂). Similar results were observed in two other experiments shown in Table 1. When the results of these three experiments are averaged, platelets treated with 9Z, 11Z-CLA stimulated M release 414±155% and enhanced TXB₂ formation 180±30%.

TABLE 1
Release of AA and TXB2 from platelets preesterified with 9Z, 11Z-CLA

| Experiment | | AA (ng/ml | Control | TXB₂ (ng/ml | Control |
|---|---|---|---|---|---|
| No. | Treatment | platelets) | (%) | platelets) | (%) |
| 1 | HSA | 5620 | 100 | 0.189 | 100 |
| | HSA + ZZ | 27000 | 480 | 0.362 | 192 |
| 2 | HSA | 311 | 100 | 0.156 | 100 |
| | HSA + ZZ | 738 | 237 | 0.317 | 203 |
| 3 | HSA | 4300 | 100 | 0.489 | 100 |
| | HSA + ZZ | 21700 | 505 | 0.713 | 146 |

| | | | |
|---|---|---|---|
| Summary | HSA + ZZ | 414 ± 155^{a} | 180 ± 30^{a} |
| #1-3 | | | |

Human platelets were treated for 1 hour at 37° C. with either HSA alone or complexed with 25 μM of 9Z, 11Z-CLA. After washing, the platelets were maintained at room temperature for 15 minutes, the supernatants were removed and after TLC, assayed for TXB₂ by EIA and free AA by HPLC.
^{a}Mean (±S.D.) is statistically different from control, P < 0.07.

### EXAMPLE 2

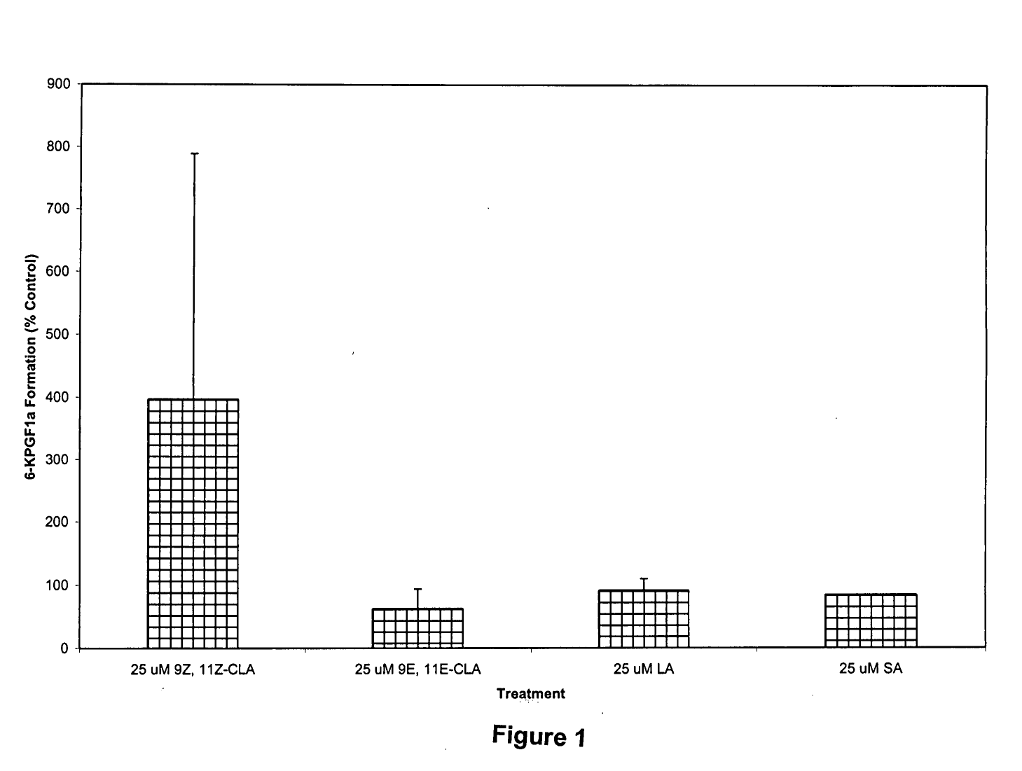

To compare the stimulatory effect of 9Z, 11Z-CLA on platelet TXB₂ formation with other fatty acids, platelets were pretreated for 1 hour with HSA (control), or HSA complexed with either 9Z, 11Z-CLA, 9E,11E-CLA, linoleic acid (LA) or stearic acid (SA). The comparative effects of 25 μM 9Z, 11Z-CLA-, 9E, 11E-CLA-, LA- or SA-treated platelets on platelet TXB₂ production is shown is FIG. 1. The values are the mean ±SD.

As shown in FIG. 1, only the 9Z, 11Z-CLA-treated platelets stimulate TXB₂ production (4-fold). On the other hand, TXB₂ formation was inhibited (37%) with platelets pretreated with the 9E, 11E-CLA isomer whereas platelets treated with either the nonconjugated LA or saturated SA did not show an appreciable effect on TXB₂ formation. These results suggests that the stimulatory effect on platelet TXB formation is quite dependent on the presence of a conjugated double bond moiety but also on the specific isomeric orientation.

### EXAMPLE 3

Human platelets were treated for 2 hours at 37° C. with either HSA complexed with 25 μM of the indicated CLA isomer or with HSA alone. After washing, the platelets were maintained at room temperature for 15 minutes. The supernatants were then removed, the lipid content extracted with chloroform/methanol and the residue, after TLC separation, assayed for TXB₂ formation by EIA. The results shown in Table 2 were similar to those obtained with CLA-prelabeled HUVECs. Prelabeling platelets with 25 μM 9Z, 11Z-CLA resulted in a 2.3 fold increase in the formation and release of TXB₂ but only a 30% increase was observed with platelets enriched with the 10,12 isomer. Also shown in Table 2 is that the CLA content of the platelet lipids increased from 0.03% to 0.23% after platelet treatment with 9Z, 11Z-CLA/HSA and to 0.66% after 10E, 12Z-CLA/HSA.

TABLE 2
CLA-prelabeling of platelets stimulates generation and release of
thromboxane B₂ and increases the CLA content of platelet lipids.

| | | CLA content | |
|---|---|---|---|
| | TXB₂ formation* | (% total lipids)** | |
| Pretreatment | Fold stimulation | 9Z, 11Z- | 10E, 12Z- |
| HSA (control) | 1 | 0.03 | 0.03 |
| +25 μM 9Z, 11Z-CLA | 2.3 ± 1.0 (4) | 0.23 | 0.03 |
| +25 μM 10E, 12Z-CLA | 1.3 ± 0.41 (4) | 0.03 | 0.66 |

*The values are the mean ± SD and were obtained from 4 separate experiments.
**Platelets were treated for 2 hours as indicated, followed by lipid extraction and transmethylation. Fatty acid methyl esters were analyzed by gas liquid chromatography.

To determine whether such stimulatory effects of these CLA isomers could also be observed with a different cell type, the interaction of the CLA isomers with human endothelial cells after enriching the endothelial cells with these fatty acids was examined.

### EXAMPLE 4

The following example illustrates the effect of CLA incorporation on 6-ketoPGF₁ₐ production from CLA-enriched HUVECs. Following the procedure described by Bordet (1990), IL-1β-treated HUVECs were first pre-enriched during an overnight incubation with human serum albumin (HSA) complexed with either 25 βM 9Z, 11Z-CLA/HSA, 25 μM 10E, 12Z-CLA/HSA or without added fatty acid (control). These isomers were chosen as the 10E, 12Z-CLA, but not 9Z, 11Z-CLA isomer, was an effective inhibitor of HUVEC 6-ketoPGF₁ₐ production. Insignificant differences in cell viabilities (as measured by LDH release) were observed between control cells and cells pretreated with HSA/CLA. After washing, the cells were incubated for 15 minutes, followed by removal of the supernatants, which were then assayed for 6-ketoPGF₁ₐ content by EIA. In four separate experiments, control HUVECs produced and released 42-1000 pg/ml of 6-ketoPGF₁ₐ. When the HUVECs were prelabeled with 9Z, 11Z-CLA, an 8-fold increase (range was 1.9-23 fold increase) in endogenous 6-ketoPGF₁ₐ formation was observed (relative to nonCLA-treated control) whereas the 10E, 12Z isomer was not quite half as effective (range was 1.5-7.5 fold increase, Table 3).

TABLE 3
CLA-prelabeling of HUVECs stimulates generation and
release of 6-ketoPGF1a.

| | |
|---|---|
| CLA-prelabeled cells | HUVEC 6-ketoPGF₁ₐ |

| | | |
|---|---|---|
| 9Z, 11Z | 10E, 12Z | formation fold- |

| | |
|---|---|
| (μM) | stimulation |

| | | |
|---|---|---|
| — | — | 1 |
| 25 | — | 8.1 ± 10 |
| — | 25 | 3.5 ± 2.7 |

The values are the mean ± SD and are obtained from 4 separate experiments.

### EXAMPLE 5

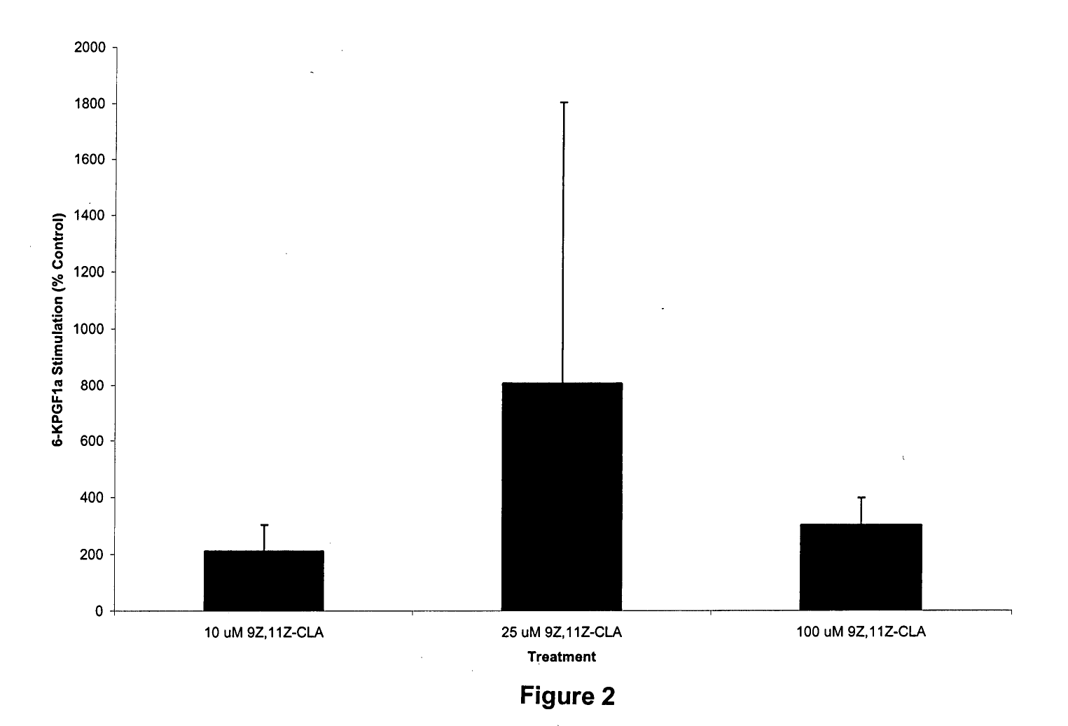

The procedure of Example 4 was used with different concentrations of 9Z, 11Z-CLA to pre-label HUVECs. The stimulatory effect on 6-ketoPGF₁ₐ formation of using different concentrations of 9Z, 11Z-CLA to pre-label HUVECs is shown in FIG. 2. The values in FIG. 2 are the mean ±SD from 3-4separate experiments.

The unusual enhancement of TXB₂ in platelets and 6-ketoPGF₁ₐ in HUVECs as a result of treatment with 9Z, 11Z-CLA may be due to an increased release of the precursor AA (Table 1) as a result of a possible stimulation of the platelet phospholipase. This result correlates with the decreased AA content in total fatty acids and in both PC and PE as a result of incorporation of 9Z, 11Z-CLA into platelets (not shown). In fact, this decrease in AA content is an order of magnitude greater than the increase in esterification observed with the 9Z, 11Z-CLA isomer, so that CLA has not simply replaced the AA.

In summary, the findings indicate that incorporation of certain CLA isomers into cellular lipids can lead to stimulation of release of cellular arachidonic acid and enhancement of prostanoid formation. Thus, (1) incorporation of 9Z, 11Z-CLA into platelet lipids resulted in a 2-5 fold stimulation of arachidonic acid release as well as a 2-4 fold stimulation of thromboxane A₂ production as assayed by its inactive metabolite thromboxane B₂ and (2) incorporation of 9Z, 11Z-CLA (and to a lesser extent, the 10 E, 12Z-CLA isomer) into HUVECs resulted in a 8-fold stimulation of prostacyclin production, as measured by its stable metabolite 6-ketoPGF₁ₐ;

References

The references mentioned earlier are more fully identified as follows:

S. F. Chin, W. Liu, J. M. Storkson, Y. L. Ha and M. W. Pariza, Dietary sources of conjugated dienoic isomers of linoleic acid, a newly recognized class of anticarcinogens, I. Food Comp. Anal., 5:185-197 (1992).

N. C. Shanta, E. A. Decker and Z. Ustunol, Conjugated linoleic acid concentration in processed cheese, J. Am. Oil Chem. Soc., 69:425-428 (1992).

C. Ip, S. F. Chin, J. A. Scimeca and M. W. Pariza, Mammary Cancer prevention by conjugated dienoic derivative of linoleic acid, Cancer Res., 51:6118-6124 (1991).

C. Ip, M. Singh, H. J. Thompson and J. A. Scimeca, Conjugated linoleic acid suppresses mammary carcinogenesis and proliferative activity of the mammary gland in the rat, Cancer Res., 54:63-128 (1992).

M. A. Belury, Conjugated dienoic linoleate: a polyunsaturated fatty acid with unique chemoprotective properties, Nutr. Rev., 53:83-89 (1995).

K. M. Lee, D. Kritchevsky and M. W. Pariza, Conjugated linoleic acid and atherosclerosis in rabbits, Atherosclerosos, 108: 19-25 (1994).

W. L. Smith and F. A. Fitzpatrick, The eicosanoids: cyclooxygenase, lipoxygenase and epoxygenase pathways in Biochemistry of Lipids, Lipoproteins and Membranes, (D. E. Vance and J. Vance Eds), Elsevier, pp. 283-308 (1996).

J. C. Bordet, M. Guichardant and M. Lagarde, Modulation of prostanoid formatin by various polyunsaturated fatty acids during platelet-endothelial cell interactions. Prostagl. Leukot. EFA 39: 197-202, 1990.

R. J. Nicolosi, E. J. Rogers, D. Kritchevsky, J. A. Scimeca and P. J. Huth, Dietary conjugated linoleic acid reduces plasma lipoproteins and early aortic atherosclerosis and hypercholesterolemic hamsters, Artery, 22: 266-277, 1997.

A. A. Spector, J. C. Hoak, G. L. Fry, G. M. Denning, L. L. Stoll and J. B. Smith, Effect of fatty acid modification of prostacyclin production by cultured human endothelial cells. J. Clin. Invest. 65: 1003-1012, 1980.

B. B. Weksler, Regulatory mechanisms in prostacyclin-blood vessel interactions, in Prostaglandins, Leukotrienes and Lipoxins, J. M. Bailey, ed. Plenum Press, New York, 1985, pp 261-266.

The purpose of the above description and examples is to illustrate some embodiments of the present invention without implying any limitation. It will be apparent to those of skill in the art that various modifications and variations may be made to the composition and method of the present invention without departing from the spirit or scope of the invention. All patents and publications cited herein are incorporated by reference in their entireties.

I claim:

1. A method of stimulating prostacyclin formation in cells, which method comprises contacting said cells with at least one conjugated linoleic acid under conditions wherein the conjugated linoleic acid becomes esterified inside the cells to form a lipid containing said at least one conjugated linoleic acid.

2. The method of claim 1, wherein said at least one conjugated linoleic acid is selected from the group consisting of 10,12-octadecadienoic acid and 9,11-octadecadienoic acid and mixtures thereof.

3. The method of claim 1, wherein the cells are endothelial cells.

4. A method of stimulating prostacyclin formation in cells, which method comprises contacting said cells with an effective amount of a ester containing at least one conjugated linoleic acid.

5. The method of claim 4, wherein said at least one conjugated linoleic acid is selected from the group consisting of 10,12-octadecadienoic acid and 9,11-octadecadienoic acid and mixtures thereof.

6. A method of stimulating prostacyclin formation in cells, which method comprises contacting said cells with an effective amount of a lipid containing at least one conjugated linoleic acid.

7. The method of claim 6, wherein said at least one conjugated linoleic acid is selected from the group consisting of 10,12-octadecadienoic acid and 9,11-octadecadienoic acid and mixtures thereof.

8. The method of claim 6 wherein the lipid is a phospholipid.

9. The method of claim 6 wherein the lipid is selected from the group consisting of phosphatidylcholine, phosphatidylethanolamine, phosphatidylinositol, phosphatidylserine, cardiolipin and sphingomyelin.

10. The method of claim 6 wherein the conjugated linoleic acid is 9Z, 11Z conjugated linoleic acid.

11. A method of stimulating prostacyclin formation in a subject, which method comprises administering to said subject an effective amount of a ester containing at least one conjugated linoleic acid.

12. The method of claim 11, wherein said at least one conjugated linoleic acid is selected from the group consisting of 10,12-octadecadienoic acid and 9,11-octadecadienoic acid and mixtures thereof.

13. The method of claim 11 wherein the ester is a lipid.

14. The method of claim 13 wherein the lipid is a phospholipid.

15. The method of claim 13 wherein the lipid is selected from the group consisting of phosphatidylcholine, phosphatidylethanolamine, phosphatidylinositol, phosphatidylserine, cardiolipin and sphingomyelin.

16. The method of claim 11 wherein the conjugated linoleic acid is 9Z,11Z conjugated linoleic acid.

17. A food composition comprising a food and an additive added to the food, wherein the additive comprises at least one ester containing at least one conjugated linoleic acid, the ester being present in an amount sufficient to assist in stimulating prostacyclin formation in a subject consuming the food composition.

18. The method of claim 17, wherein said at least one conjugated linoleic acid is selected from the group consisting of 10,12-octadecadienoic acid and 9,11-octadecadienoic acid and mixtures thereof.

19. The method of claim 17 wherein the ester is a lipid.

20. The method of claim 19 wherein the lipid is a phospholipid.

21. The method of claim 19 wherein the lipid is selected from the group consisting of phosphatidylcholine, phosphatidylethanolamine, phosphatidylinositol, phosphatidylserine, cardiolipin and sphingomyelin.

22. The method of claim 17 wherein the conjugated linoleic acid is 9Z,11Z conjugated linoleic acid.

23. A pharmaceutical composition in tablet or capsule form for use in stimulating prostacyclin formation which comprises, as the active component, an effective amount of at least one ester containing at least one conjugated linoleic acid, together with a pharmaceutically acceptable carrier.

24. The method of claim 23, wherein said at least one conjugated linoleic acid is selected from the group consisting of 10,12-octadecadienoic acid and 9,11-octadecadienoic acid and mixtures thereof.

25. The method of claim 23 wherein the ester is a lipid.

26. The method of claim 25 wherein the lipid is a phospholipid.

27. The method of claim 23 wherein the conjugated linoleic acid is 9Z, 11Z conjugated linoleic acid.

28. The method of claim 25 wherein the lipid is selected from the group consisting of phosphatidylcholine, phosphatidylethanolamine, phosphatidylinositol, phosphatidylserine, cardiolipin and sphingomyelin.

29. A method of stimulating thromboxane formation in cells, which method comprises contacting said cells with 9Z,11Z octadecadienoic acid under conditions wherein the 9Z,11Z octadecadienoic acid becomes esterified inside the cells to form a lipid containing 9Z,11Z octadecadienoic acid.

30. The method of claim 29 wherein the cells are platelets.

31. A method of stimulating thromboxane formation in cells, which method comprises contacting said cells with an effective amount of an ester of 9Z,11Z octadecadienoic acid.

32. The method of claim 31 wherein the ester is a lipid is selected from the group consisting of phosphatidylcholine, phosphatidylethanolamine, phosphatidylinositol, phosphatidylserine, cardiolipin and sphingomyelin.

33. A method of stimulating thomboxane formation in a subject, which method comprises administering to said subject an effective amount of an ester of 9Z,11Z octadecadienoic acid.

34. The method of claim 33 wherein the ester is a lipid is selected from the group consisting of phosphatidylcholine, phosphatidylethanolamine, phosphatidylinositol, phosphatidylserine, cardiolipin and sphingomyelin.

35. A food composition comprising a food and an additive, the additive comprising an ester of 9Z,11Z octadecadienoic acid, the ester being present in an amount sufficient to assist in stimulating thomboxane formation in a subject consuming the food composition.

36. A pharmaceutical composition in tablet or capsule form for use in stimulating thromboxane formation which comprises, as the active component, an effective amount of an ester of 9Z, 11Z octadecadienoic acid, together with a pharmaceutically acceptable carrier.

37. A method of stimulating a release of arachidonic acid in cells, which method comprises contacting said cells with 9Z, 11Z octadecadienoic acid under conditions wherein the 9Z, 11Z octadecadienoic acid becomes esterified inside the cells to form a lipid containing 9Z, 11Z octadecadienoic acid.

38. The method of claim 37 wherein the cells are platelets or endothelial cells.

39. A method of stimulating a release of arachidonic acid in cells, which method comprises contacting said cells with an effective amount of an ester of 9Z, 11Z octadecadienoic acid.

40. The method of claim 39 wherein the ester is a lipid is selected from the group consisting of phosphatidylcholine, phosphatidylethanolamine, phosphatidylinositol, phosphatidylserine, cardiolipin and sphingomyelin.

41. A method of stimulating a release of arachidonic acid in a subject, which method comprises administering to said subject an effective amount of an ester of 9Z, 11Z octadecadienoic acid.

42. The method of claim 41 wherein the ester is a lipid is selected from the group consisting of phosphatidylcholine, phosphatidylethanolamine, phosphatidylinositol, phosphatidylserine, cardiolipin and sphingomyelin.

43. A food composition comprising a food and an additive, the additive added thereto, the additive comprising an ester of 9Z, 11Z octadecadienoic acid, the ester being present in an amount sufficient to assist in stimulating a release of arachidonic acid in a subject consuming the food composition.

44. A pharmaceutical composition in tablet or capsule form for use in stimulating a release of arachidonic acid which comprises, as the active component, an effective amount of an ester of 9Z, 11Z octadecadienoic acid, together with a pharmaceutically acceptable carrier.
